(19) United States
(12) Patent Application Publication
(10) Pub. No.: US 2020/0387342 A1
(43) Pub. Date: Dec. 10, 2020

(54) INFORMATION PROCESSING DEVICE AND NON-TRANSITORY COMPUTER READABLE MEDIUM

(71) Applicant: FUJI XEROX CO., LTD., Tokyo (JP)

(72) Inventors: Kosuke AOKI, Kanagawa (JP); Tsutomu KIMURA, Kanagawa (JP); Tadashi SUTO, Kanagawa (JP)

(73) Assignee: FUJI XEROX CO., LTD., Tokyo (JP)

(21) Appl. No.: 16/658,575

(22) Filed: Oct. 21, 2019

(30) Foreign Application Priority Data
Jun. 6, 2019 (JP) 2019-105784

(51) Int. Cl. G06F 3/16; G06F 3/01; G10L 25/51; A61B 5/00; A61B 5/11; H04R 29/00

(52) U.S. Cl. CPC G06F 3/165; G06F 3/012; G06F 3/015; H04B 1/3827; A61B 5/6803; A61B 5/11; H04R 29/004; G10L 25/51

(57) ABSTRACT

An information processing device includes a first obtaining unit that obtains action information from a device worn on a head of a user, the action information being information indicating a motion of the head of the user; a second obtaining unit that obtains biological information on the user from the device; and an analyzing unit that analyzes, based on the action information and the biological information, a state of the user.

### CROSS-REFERENCE TO RELATED APPLICATIONS

This application is based on and claims priority under 35 USC 119 from Japanese Patent Application No. 2019-105784 filed Jun. 6, 2019.

### BACKGROUND

### (i) Technical Field

The present disclosure relates to an information processing device and a non-transitory computer readable medium.

### (ii) Related Art

Japanese Unexamined Patent Application Publication No. 2016-118575 is directed to providing a device capable of estimating an intracerebral intellectual activity state of a user who is performing an activity by using an interface device, and discloses an intracerebral intellectual activity estimating device. The intracerebral intellectual activity estimating device is connected to an interface device that receives/outputs intellectual activity information from/to a user and that is capable of processing the intellectual activity information. The intracerebral intellectual activity estimating device includes a brain wave analyzing unit that generates a brain wave analysis log by recording in time series brain wave information based on brain wave data obtained from the user; a brain wave interpreting unit that determines, based on plural interpretation rules in which chronological data of the brain wave information is associated in advance with interpretation labels of an intracerebral intellectual activity, a candidate interpretation label from the generated brain wave analysis log; an activity status grasping unit that determines, based on a processing status of the intellectual activity information in the interface device, a candidate state label from among plural state labels of the intracerebral intellectual activity set in advance; and an intellectual activity determining unit that determines content of a label common between the determined candidate interpretation label and the determined candidate state label to be an intracerebral intellectual activity state of the user.

Japanese Unexamined Patent Application Publication No. 2019-022540 is directed to objectively grasping stress on a caregiver from work, and discloses an information processing device. The information processing device obtains brain wave data of an evaluation target detected by using a wearable sensor, determines whether the obtained brain wave data is a normal value or abnormal value by referring to a storage unit storing brain wave data specified in advance as a normal value or abnormal value, transmits an inquiry asking whether or not there is stress to an information processing terminal corresponding to the evaluation target in a case where it is determined that the brain wave data is an abnormal value, and corrects a determination result of the brain wave data in response to receipt of an answer to the inquiry.

### SUMMARY

In the case of analyzing a state of a user by using biological information and action information on the user in combination, it is difficult to analyze the state of the user if a device that detects a biological state of the user is different from a device that obtains action information other than biological information.

Aspects of non-limiting embodiments of the present disclosure relate to an information processing device and a non-transitory computer readable medium that are capable of obtaining action information and biological information from a device worn on the head of a user and analyzing a state of the user.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided an information processing device including a first obtaining unit that obtains action information from a device worn on a head of a user, the action information being information indicating a motion of the head of the user; a second obtaining unit that obtains biological information on the user from the device; and an analyzing unit that analyzes, based on the action information and the biological information, a state of the user.

### BRIEF DESCRIPTION OF THE DRAWINGS

An exemplary embodiment of the present disclosure will be described in detail based on the following figures, wherein:

FIG. 1 is a conceptual module configuration diagram illustrating an example configuration according to the exemplary embodiment;

FIG. 2 is an explanatory diagram illustrating an example system configuration utilizing the exemplary embodiment;

FIG. 3 is an explanatory diagram illustrating an example of actual usage according to the exemplary embodiment;

FIG. 4 is a flowchart illustrating an example of processing according to the exemplary embodiment;

FIG. 5 is an explanatory diagram illustrating an example data structure of a state estimation table;

FIG. 6 is an explanatory diagram illustrating an example data structure of a feedback table;

FIG. 7 is a flowchart illustrating an example of processing according to the exemplary embodiment;

FIG. 8 is an explanatory diagram illustrating an example data structure of a location estimation table;

FIG. 9 is an explanatory diagram illustrating an example data structure of a feedback table;

FIG. 10 is a flowchart illustrating an example of processing according to the exemplary embodiment;

FIG. 11 is a flowchart illustrating an example of processing according to the exemplary embodiment; and

FIG. 12 is a block diagram illustrating an example hardware configuration of a computer that implements the exemplary embodiment.

### DETAILED DESCRIPTION

Hereinafter, an exemplary embodiment for carrying out the present disclosure will be described with reference to the attached drawings.

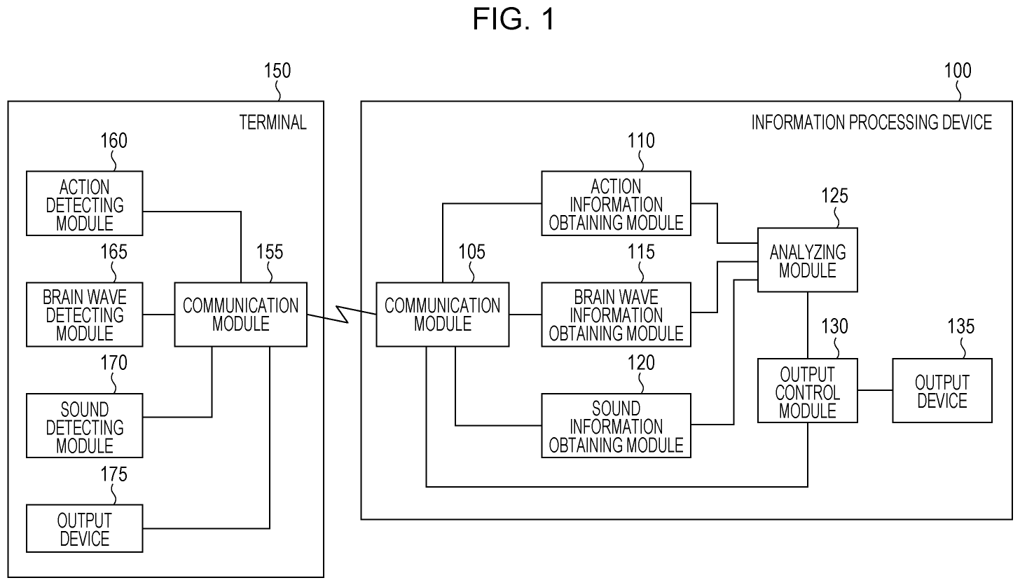

FIG. 1 is a conceptual module configuration diagram of an example configuration according to the exemplary embodiment.

Modules are components of software (including computer programs as the interpretation of “software”) or hardware that can be logically separated from one another in general. Thus, the modules according to the exemplary embodiment include not only modules in a computer program but also modules in a hardware configuration. Therefore, the description of the exemplary embodiment includes a description of a computer program for causing a computer to function as those modules (for example, a program for causing a computer to execute individual steps, a program for causing a computer to function as individual units, or a program for causing a computer to implement individual functions), a system, and a method. For the convenience of description, “store”, “cause . . . to store”, or an expression equivalent thereto may be used. These expressions mean “cause a storage device to store” or “perform control to cause a storage device to store” in a case where an exemplary embodiment is a computer program. The modules may correspond to functions on a one-to-one basis. In terms of packaging, a single module may be constituted by a single program, plural modules may be constituted by a single program, or a single module may be constituted by plural programs. Plural modules may be executed by a single computer, or a single module may be executed by plural computers in a distributed or parallel environment. Alternatively, a single module may include another module. Hereinafter, the term “connection” will be used to refer to a logical connection (for example, transmission and reception of data, instructions, a referential relationship between pieces of data, login, etc.) as well as a physical connection. The term “predetermined” means being determined before target processing, and includes the meaning of being determined in accordance with a present situation/state or a previous situation/state, before target processing before or after processing according to the exemplary embodiment starts. In a case where there are plural “predetermined values”, the plural predetermined values may be different from one another, or two or more of the values (of course including all the values) may be the same. A description “in the case of A, B is performed” is used as the meaning “whether A or not is determined, and B is performed if it is determined A”, except for a case where the determination of whether A or not is unnecessary. Enumeration of items, such as “A, B, and C”, is merely enumeration of examples unless otherwise noted, and includes selection of only one of them (for example, only A).

A system or device may be constituted by plural computers, hardware units, devices, or the like connected to one another through a communication medium, such as a network (“network” includes communication connections on a one-to-one basis), or may be constituted by a single computer, hardware unit, device, or the like. The terms “Device” and “system” are used synonymously. Of course, “system” does not include a man-made social “organization” (i.e., a social system).

Target information is read from a storage device in individual processing operations performed by respective modules or in individual processing operations when plural processing operations are performed within a module. After each processing operation is performed, a result of the processing is written into the storage device. Thus, a description of reading from the storage device before a processing operation and writing into the storage device after a processing operation may be omitted. Examples of the storage device include a hard disk drive, a random access memory (RAM), an external storage medium, a storage device connected through a communication line, a register in a central processing unit (CPU), and the like.

An information processing device 100 according to the exemplary embodiment has a function of estimating a state of a user by using biological information or the like on the user and includes, as illustrated in the example in FIG. 1, a communication module 105, an action information obtaining module 110, a brain wave information obtaining module 115, a sound information obtaining module 120, an analyzing module 125, an output control module 130, and an output device 135.

The “biological information” herein is information obtained by measuring a vital activity of a human body. Examples of the biological information include information on an electrocardiogram, heart rate, blood pressure, body temperature, brain wave, myoelectric potential, and retinal (fundus) potential. In the exemplary embodiment, brain wave information is mainly used as an example.

With use of brain wave information as biological information and motion information on a user, a state of the user is estimated.

A terminal 150 is a device worn on the head of a user (“wear” includes the concept of “put on”). The user herein is a person who is using the information processing device 100. That is, a person who is using the information processing device 100 is identical to a person who is wearing the terminal 150. The terminal 150 is worn on the head of the user and incorporates at least a sensor capable of detecting a motion of the head of the user and a brain wave of the user. The terminal 150 is a so-called wearable device.

The communication module 105 is connected to the action information obtaining module 110, the brain wave information obtaining module 115, the sound information obtaining module 120, and the output control module 130, and is also connected to a communication module 155 of the terminal 150 through a communication line. The communication module 105 communicates with the terminal 150. The communication herein may be performed in a wireless or wired manner. For example, Wi-Fi, Bluetooth (registered trademark), Universal Serial Bus (USB), or the like may be used. The communication module 105 transfers data received from the terminal 150 to the action information obtaining module 110, the brain wave information obtaining module 115, or the sound information obtaining module 120, and transmits data received from the output control module 130 to the terminal 150.

The action information obtaining module 110 is connected to the communication module 105 and the analyzing module 125. The action information obtaining module 110 obtains action information, which is information indicating a motion of the head of the user, from the terminal 150 through the communication module 105.

An example of the “action information” herein is information detected by an acceleration sensor. As the acceleration sensor, for example, a six-axis acceleration sensor capable of detecting accelerations along three axes and angular velocities along three axes may be used.

The brain wave information obtaining module 115 is connected to the communication module 105 and the analyzing module 125. The brain wave information obtaining module 115 obtains brain wave information, which is information indicating a brain wave of the user, from the terminal 150 through the communication module 105.

An example of the “brain wave information” herein is information detected by a myoelectric sensor.

The sound information obtaining module 120 is connected to the communication module 105 and the analyzing module 125. The sound information obtaining module 120 obtains sound information, which is information indicating a sound produced by the user or a sound from surroundings of the user, from the terminal 150 through the communication module 105. Voice is an example of a sound.

The terminal 150 further includes a sound detecting module 170. The sound detecting module 170 is, for example, a microphone.

The analyzing module 125 is connected to the action information obtaining module 110, the brain wave information obtaining module 115, the sound information obtaining module 120, and the output control module 130. The analyzing module 125 analyzes, based on the action information obtained by the action information obtaining module 110 and the brain wave information obtained by the brain wave information obtaining module 115, a state of the user.

Alternatively, the analyzing module 125 may analyze, based on the action information obtained by the action information obtaining module 110, the brain wave information obtained by the brain wave information obtaining module 115, and the sound information obtained by the sound information obtaining module 120, a state of the user.

The analyzing module 125 may obtain a schedule of the user. In a case where the sound information obtaining module 120 does not obtain sound information at a time when a sound is supposed to be produced according to the schedule, the analyzing module 125 may analyze that there is a possibility that the sound detecting module 170 included in the terminal 150 has a failure.

The “time when a sound is supposed to be produced according to the schedule” herein includes at least the time when the user works with another person, for example, in a meeting or consultation.

In a case where the action information obtained by the action information obtaining module 110 does not include information indicating a shake of the head, the analyzing module 125 may analyze that there is a possibility that an action detecting module 160, which is a motion detecting sensor included in the terminal 150, has a failure.

Furthermore, in a case where the analyzing module 125 analyzes that there is a possibility that the action detecting module 160 has a failure, the analyzing module 125 may analyze a state of the user, based on the sound information obtained by the sound information obtaining module 120 and the brain wave information obtained by the brain wave information obtaining module 115.

The output control module 130 is connected to the communication module 105, the analyzing module 125, and the output device 135. The output control module 130 performs control to output information to an output device 175 included in the terminal 150 in accordance with the state of the user analyzed by the analyzing module 125.

The output control module 130 may perform control to cause sound information corresponding to the state of the user to be output from the output device 175. Specifically, this is processing performed in a case where a headphone, an earphone, a speaker, or the like is adopted as the output device 175.

The sound information output from a speaker or the like serving as the output device 175 of the terminal 150 is information that indicates the state of the user and that is fed back to the user. Examples of the sound information include a sound reporting the state of the user, and music for improving, maintaining, or degrading the state of the user.

The output control module 130 may perform control to output information not only to the output device 175 but also to the output device 135 in accordance with the state of the user analyzed by the analyzing module 125. In this case, the information to be output may be sound information or display information of characters, figures, graphs, images, or the like.

The output device 135 is connected to the output control module 130. The output device 135 outputs an analysis result of the analyzing module 125, feedback information, or the like to a display device, such as a liquid crystal display or an organic electroluminescence (EL) display, or a speaker or the like, in accordance with control by the output control module 130. The user of the information processing device 100 (the user wearing the terminal 150) is capable of knowing his/her state and receiving feedback.

The terminal 150 includes the communication module 155, the action detecting module 160, a brain wave detecting module 165, the sound detecting module 170, and the output device 175.

The communication module 155 is connected to the action detecting module 160, the brain wave detecting module 165, the sound detecting module 170, and the output device 175, and is also connected to the communication module 105 of the information processing device 100 through the communication line. The communication module 155 communicates with the information processing device 100. The communication herein may be performed in a wireless or wired manner. For example, Wi-Fi, Bluetooth (registered trademark), USB, or the like may be used. The communication module 155 transmits data received from the action detecting module 160, the brain wave detecting module 165, or the sound detecting module 170 to the information processing device 100, and transmits data received from the information processing device 100 to the output device 175.

The action detecting module 160 is connected to the communication module 155. The action detecting module 160 detects a motion of the head of the user wearing the terminal 150.

The brain wave detecting module 165 is connected to the communication module 155. The brain wave detecting module 165 detects a brain wave of the user wearing the terminal 150.

The sound detecting module 170 is connected to the communication module 155. The sound detecting module 170 is a microphone that detects a sound produced by the user wearing the terminal 150 or a sound from the surroundings of the user.

The output device 175 is connected to the communication module 155. The output device 175 outputs information received by the communication module 155. For example, in a case where sound information transmitted by the output control module 130 of the information processing device 100 is received, the output device 175 outputs the sound information as a sound by using a headphone, an earphone, a speaker, or the like.

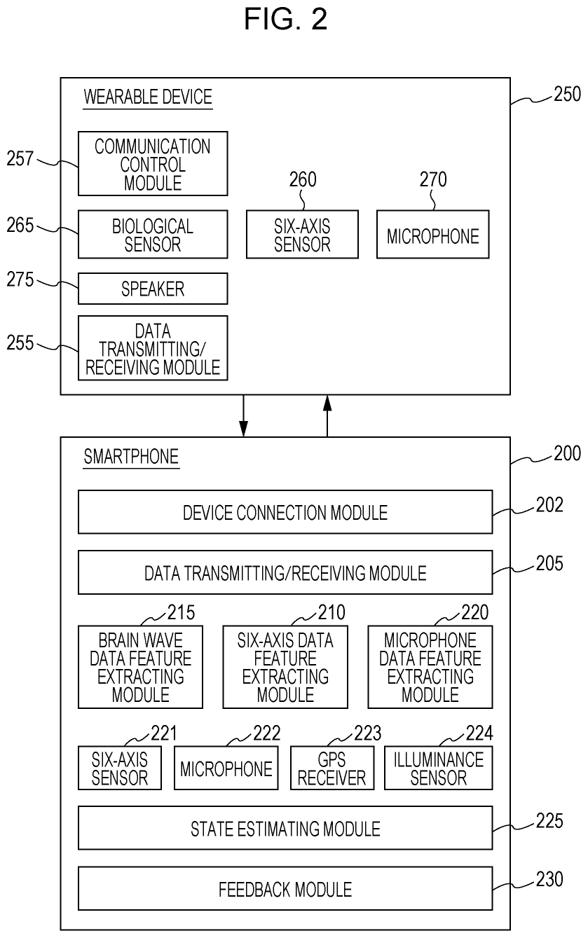

FIG. 2 is an explanatory diagram illustrating an example system configuration utilizing the exemplary embodiment.

A smartphone 200 and a wearable device 250 are connected to each other through a communication line.

The smartphone 200 includes a device connection module 202, a data transmitting/receiving module 205, a six-axis data feature extracting module 210, a brain wave data feature extracting module 215, a microphone data feature extracting module 220, a six-axis sensor 221, a microphone 222, a global positioning system (GPS) receiver 223, an illuminance sensor 224, a state estimating module 225, and a feedback module 230.

The smartphone 200 is a specific example of the information processing device 100. The device connection module 202 and the data transmitting/receiving module 205 correspond to the communication module 105, the six-axis data feature extracting module 210 corresponds to the action information obtaining module 110, the brain wave data feature extracting module 215 corresponds to the brain wave information obtaining module 115, the microphone data feature extracting module 220 corresponds to the sound information obtaining module 120, the state estimating module 225 corresponds to the analyzing module 125, and the feedback module 230 corresponds to the output control module 130.

The device connection module 202 performs connection processing (preprocessing for communication) for enabling the smartphone 200 and the wearable device 250 to communicate with each other.

The data transmitting/receiving module 205 transmits/receives data to/from the wearable device 250 after the device connection module 202 has completed the connection processing for the wearable device 250.

The six-axis data feature extracting module 210 receives data detected by a six-axis sensor 260 of the wearable device 250 and extracts a feature of the data.

The brain wave data feature extracting module 215 receives data detected by a biological sensor 265 of the wearable device 250 and extracts a feature of the data.

The microphone data feature extracting module 220 receives data detected by a microphone 270 of the wearable device 250 and extracts a feature of the data.

The smartphone 200 includes the six-axis sensor 221, the microphone 222, the GPS receiver 223, the illuminance sensor 224, and so forth, and is thus capable of detecting a state of the user carrying the smartphone 200 or a state of the surroundings of the user.

The six-axis sensor 221 is a sensor capable of detecting a movement direction, orientation, and rotation of the smartphone 200 (i.e., the user carrying the smartphone 200) and calculating a movement distance, a movement speed, and the like. The six-axis sensor 221 is formed by combining an acceleration sensor capable of detecting three directions including a forward-backward direction, a right-left direction, and an upward-downward direction and a geomagnetic sensor capable of detecting north, south, east, and west, or by combining the acceleration sensor and a gyro sensor capable of detecting a rotation speed.

The microphone 222 detects a sound produced by the user carrying the smartphone 200 or a sound from the surroundings of the user.

The GPS receiver 223 detects the position of the smartphone 200 (i.e., the position of the user carrying the smartphone 200).

The illuminance sensor 224 detects the brightness of the surroundings of the smartphone 200.

The state estimating module 225 estimates a state of the user by using processing results of the six-axis data feature extracting module 210, the brain wave data feature extracting module 215, and the microphone data feature extracting module 220, and detection results of the six-axis sensor 221, the microphone 222, the GPS receiver 223, the illuminance sensor 224, and the like.

The feedback module 230 feeds back sound information or the like to the user in accordance with the state of the user estimated by the state estimating module 225.

The wearable device 250 includes a data transmitting/receiving module 255, a communication control module 257, the six-axis sensor 260, the biological sensor 265, the microphone 270, and a speaker 275.

The wearable device 250 is a specific example of the terminal 150. The data transmitting/receiving module 255 and the communication control module 257 correspond to the communication module 155, the six-axis sensor 260 corresponds to the action detecting module 160, the biological sensor 265 corresponds to the brain wave detecting module 165, the microphone 270 corresponds to the sound detecting module 170, and the speaker 275 corresponds to the output device 175. A specific example of the wearable device 250 may be the brain wave measuring device described in Japanese Unexamined Patent Application Publication No. 2019-024758.

The data transmitting/receiving module 255 transmits/receives data to/from the smartphone 200 in accordance with control of the communication control module 257.

The communication control module 257 controls the data transmitting/receiving module 255 to communicate with the smartphone 200.

The six-axis sensor 260 is equivalent to the six-axis sensor 221 of the smartphone 200, and is capable of detecting a movement direction, orientation, and rotation of the wearable device 250 (i.e., the user wearing the wearable device 250) and calculating a movement distance, a movement speed, and the like.

The biological sensor 265 measures a brain wave of the user wearing the wearable device 250. For example, the electrode described in Japanese Unexamined Patent Application Publication No. 2019-024758 (an electrode that is made of a forming material, has conductivity at least in the portion to be in contact with a living body, and detects a brain wave while in contact with a living body) may be used.

The microphone 270 detects a sound produced by the user wearing the wearable device 250 or a sound from the surroundings of the user.

The speaker 275 outputs sound information as a sound by using a headphone, an earphone, a speaker, or the like.

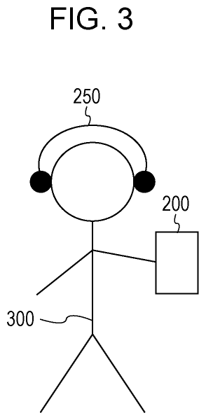

FIG. 3 is an explanatory diagram illustrating an example of actual usage according to the exemplary embodiment.

A user 300 carries the smartphone 200 and is wearing the wearable device 250 on the head. Information on a brain wave or the like of the user 300 detected by the wearable device 250 is transmitted to the smartphone 200, and the smartphone 200 analyzes the state of the user 300. With use of the display, speaker, or the like of the smartphone 200, or the speaker or the like of the wearable device 250, feedback is performed in real time in accordance with a current state of the user 300. For example, music for enhancing the concentration of the user 300, music for maintaining a relaxed state, or the like is output.

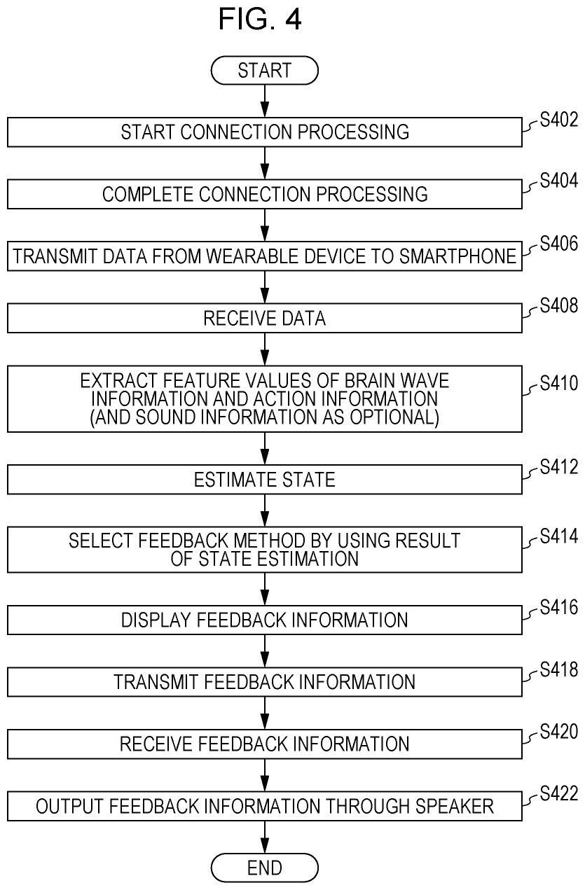

FIG. 4 is a flowchart illustrating an example of processing according to the exemplary embodiment. The processing from step S402 to step S404 is connection processing between the wearable device 250 and the smartphone 200. The processing from step S408 to step S418 is processing performed by the smartphone 200, and the processing from step S420 to step S422 is feedback processing performed by the wearable device 250.

In step S402, the smartphone 200 starts connection processing of connecting to the wearable device 250.

In step S404, the connection processing between the wearable device 250 and the smartphone 200 is completed.

In step S406, the wearable device 250 transmits data to the smartphone 200. The wearable device 250 transmits at least brain wave information detected by the biological sensor 265 and action information detected by the six-axis sensor 260. The wearable device 250 may further transmit sound information detected by the microphone 270.

In step S408, the smartphone 200 receives data.

In step S410, the smartphone 200 extracts at least a feature value of the brain wave information detected by the biological sensor 265 and a feature value of the action information detected by the six-axis sensor 260. The smartphone 200 may further extract a feature value of the sound information detected by the microphone 270. Examples of the feature value include a dominant wave (an alpha wave, a beta wave, etc.) or the like for brain wave information; “head shakes back and forth”, “head hardly moves”, or the like for action information; and “there is an utterance of user 300”, “there is no utterance of user 300”, or the like for sound information. Whether an utterance is the utterance of the user 300 or the utterance of a person other than the user 300 may be determined by using the volume of a sound detected by the microphone 270 or by using a directional microphone.

In step S412, the smartphone 200 estimates the state of the user 300 by using the extraction result obtained in step S410. For example, the smartphone 200 estimates the state by using a state estimation table 500, which will be described below.

In step S414, the smartphone 200 selects a feedback method by using a result of the state estimation performed in step S412. For example, the smartphone 200 selects a feedback method by using a feedback table 600, which will be described below.

In step S416, the smartphone 200 displays feedback information on the display device of the smartphone 200.

In step S418, the smartphone 200 transmits the feedback information to the wearable device 250.

In step S420, the wearable device 250 receives the feedback information.

In step S422, the wearable device 250 outputs the feedback information through the speaker 275.

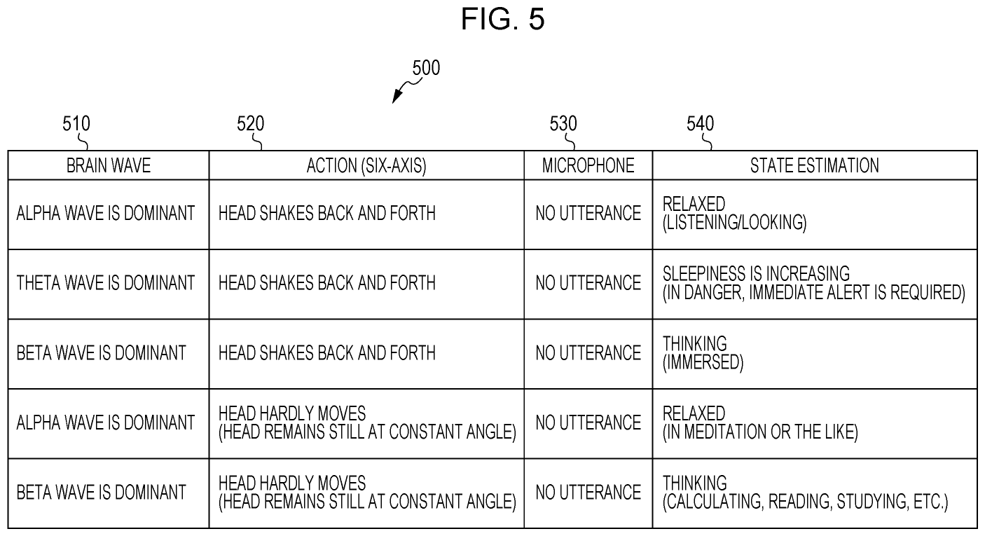

FIG. 5 is an explanatory diagram illustrating an example data structure of the state estimation table 500.

The state estimation table 500 includes a brain wave column 510, an action (six-axis) column 520, a microphone column 530, and a state estimation column 540. The brain wave column 510 stores brain wave data. The action (six-axis) column 520 stores action data (six-axis). The microphone column 530 stores microphone data. The state estimation column 540 stores state estimation data.

For example, as shown in the example in the first row of the state estimation table 500, in a case where the brain wave column 510 indicates “alpha wave is dominant”, the action (six-axis) column 520 indicates “head shakes back and forth”, and the microphone column 530 indicates “no utterance”, the state is estimated to be “relaxed (listening/looking)” in the state estimation column 540.

As shown in the example in the second row, in a case where the brain wave column 510 indicates “theta wave is dominant”, the action (six-axis) column 520 indicates “head shakes back and forth”, and the microphone column 530 indicates “no utterance”, the state is estimated to be “sleepiness is increasing (in danger, immediate alert is required)” in the state estimation column 540.

As shown in the example in the third row, in a case where the brain wave column 510 indicates “beta wave is dominant”, the action (six-axis) column 520 indicates “head shakes back and forth”, and the microphone column 530 indicates “no utterance”, the state is estimated to be “thinking (immersed)” in the state estimation column 540.

As shown in the example in the fourth row, in a case where the brain wave column 510 indicates “alpha wave is dominant”, the action (six-axis) column 520 indicates “head hardly moves (head remains still at constant angle)”, and the microphone column 530 indicates “no utterance”, the state is estimated to be “relaxed (in meditation or the like)” in the state estimation column 540.

As shown in the example in the fifth row, in a case where the brain wave column 510 indicates “beta wave is dominant”, the action (six-axis) column 520 indicates “head hardly moves (head remains still at constant angle)”, and the microphone column 530 indicates “no utterance”, the state is estimated to be “thinking (calculating, reading, studying, etc.)” in the state estimation column 540.

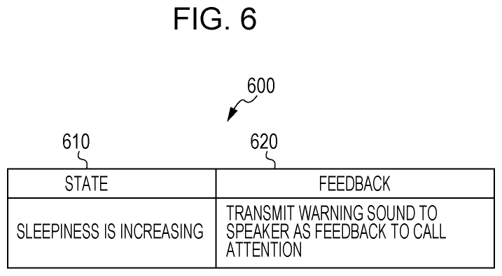

FIG. 6 is an explanatory diagram illustrating an example data structure of the feedback table 600.

The feedback table 600 includes a state column 610 and a feedback column 620. The state column 610 stores state data. The feedback column 620 stores feedback data.

For example, in a case where the state is “sleepiness is increasing”, “transmit warning sound to speaker as feedback to call attention” is performed as feedback.

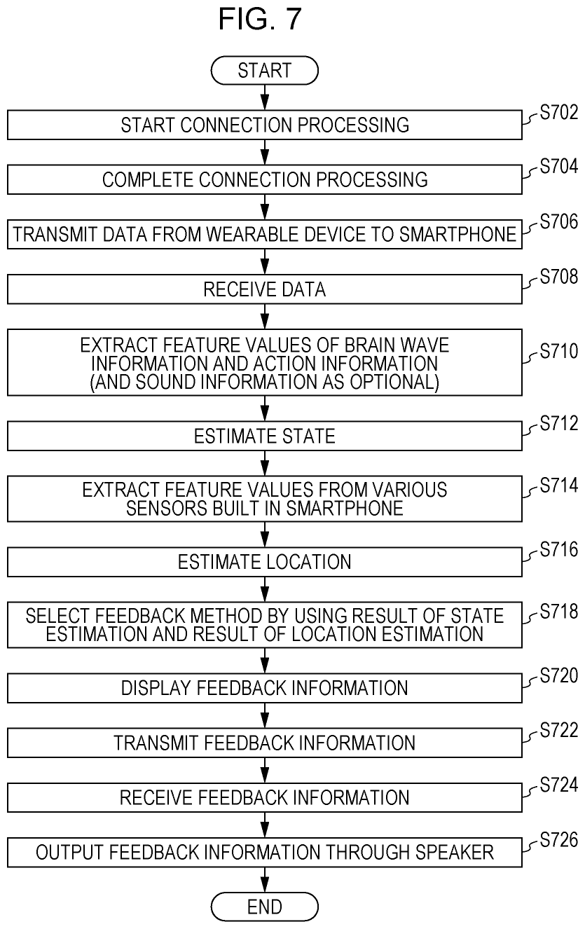

FIG. 7 is a flowchart illustrating an example of processing according to the exemplary embodiment. The processing from step S702 to step S704 is connection processing between the wearable device 250 and the smartphone 200. The processing from step S708 to step S722 is processing performed by the smartphone 200, and the processing from step S724 to step S726 is feedback processing performed by the wearable device 250.

In step S702, the smartphone 200 starts connection processing of connecting to the wearable device 250.

In step S704, the connection processing between the wearable device 250 and the smartphone 200 is completed.

In step S706, the wearable device 250 transmits data to the smartphone 200. The wearable device 250 transmits at least brain wave information detected by the biological sensor 265 and action information detected by the six-axis sensor 260. The wearable device 250 may further transmit sound information detected by the microphone 270.

In step S708, the smartphone 200 receives data.

In step S710, the smartphone 200 extracts at least a feature value of the brain wave information detected by the biological sensor 265 and a feature value of the action information detected by the six-axis sensor 260. The smartphone 200 may further extract a feature value of the sound information detected by the microphone 270. Examples of the feature value include a dominant wave (an alpha wave, a beta wave, etc.) or the like for brain wave information; “head shakes back and forth”, “head hardly moves”, or the like for action information; and “there is an utterance of user 300”, “there is no utterance of user 300”, or the like for sound information. Whether an utterance is the utterance of the user 300 or the utterance of a person other than the user 300 may be determined by using the volume of a sound detected by the microphone 270 or by using a directional microphone.

In step S712, the smartphone 200 estimates the state of the user 300 by using the extraction result obtained in step S710. For example, the smartphone 200 estimates the state by using the state estimation table 500 described above.

In step S714, the smartphone 200 extracts feature values from various sensors built in the smartphone 200, such as the six-axis sensor 221 and the microphone 222. Examples of the feature values include “home”, “office”, “being out”, and the like as position information detected by the GPS receiver 223. Specifically, predetermined map information (a table showing the correspondence between information indicating a latitude, longitude, and altitude and a home, office, or the like) may be used to extract a feature value. Other examples of the feature values include “stationary”, “walking”, and the like as action information detected by the six-axis sensor 221; “light”, “dark”, and the like representing an illuminance level as illuminance information detected by the illuminance sensor 224; and “quiet”, “noisy”, and the like representing a noise level as sound information detected by the microphone 222. That is, the state of the user 300 or the state of the environment around the user 300 is detected by using the six-axis sensor 221, the microphone 222, the GPS receiver 223, the illuminance sensor 224, and the like in the smartphone 200. Specifically, as the state of the user 300, information indicating whether the user 300 is performing an action such as walking or is stationary can be obtained from detection information obtained by the six-axis sensor 221. Also, as the state of the environment around the user 300, noise information on the environment around the user 300 can be obtained from detection information obtained by the microphone 222, the location of the user 300 can be obtained from detection information obtained by the GPS receiver 223, and an illumination state of the location of the user 300 can be obtained from detection information obtained by the illuminance sensor 224.

In step S716, the smartphone 200 estimates the location of the user 300 by using the feature values extracted in step S714. For example, the smartphone 200 estimates the location by using a location estimation table 800, which will be described below.

In step S718, the smartphone 200 selects a feedback method by using a result of the state estimation in step S712 and a result of the location estimation in step S716. For example, the smartphone 200 selects a feedback method by using a feedback table 900, which will be described below. That is, in this step, the smartphone 200 analyzes the state of the user 300 and selects a feedback method, based on the state of the user 300 or the state of the environment around the user 300 estimated by using the action information and biological information obtained from the wearable device 250 and the pieces of information obtained from the various sensors in the smartphone 200.

In step S720, the smartphone 200 displays feedback information on the display device of the smartphone 200. The feedback information herein may be text information or the like indicating the state of the user 300.

In step S722, the smartphone 200 transmits feedback information to the wearable device 250. The feedback information herein may be sound information, such as music.

In step S724, the wearable device 250 receives the feedback information.

In step S726, the wearable device 250 outputs the feedback information through the speaker 260.

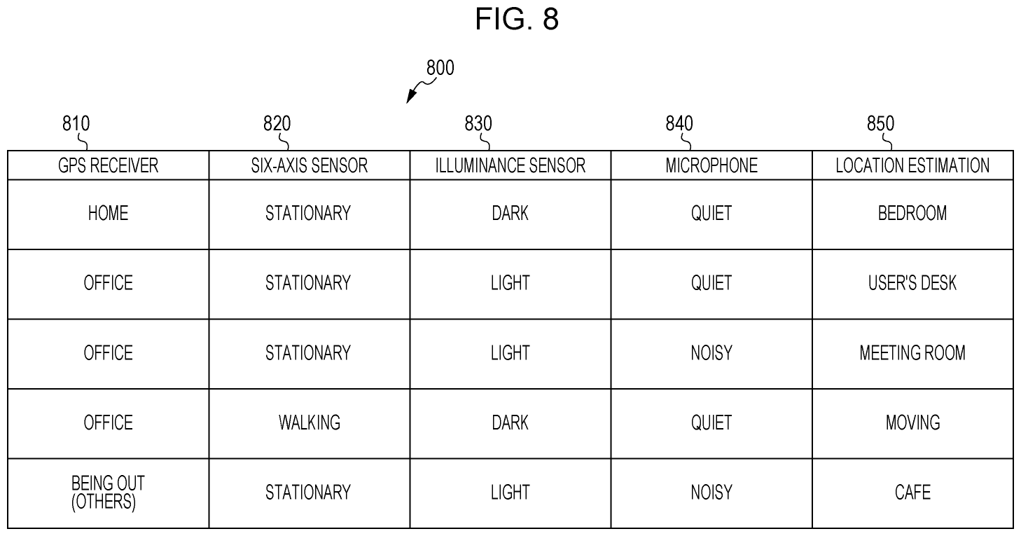

FIG. 8 is an explanatory diagram illustrating an example data structure of the location estimation table 800.

The location estimation table 800 includes a GPS column 810, a six-axis sensor column 820, an illuminance sensor column 830, a microphone column 840, and a location estimation column 850. The GPS column 810 stores GPS data. The six-axis sensor column 820 stores six-axis sensor data. The illuminance sensor column 830 stores illuminance sensor data. The microphone column 840 stores microphone data. The location estimation column 850 stores location estimation data.

For example, as shown in the example in the first row of the location estimation table 800, in a case where the GPS column 810 indicates “home”, the six-axis sensor column 820 indicates “stationary”, the illuminance sensor column 830 indicates “dark”, and the microphone column 840 indicates “quiet”, the location is estimated to be “bedroom” in the location estimation column 850.

As shown in the example in the second row, in a case where the GPS column 810 indicates “office”, the six-axis sensor column 820 indicates “stationary”, the illuminance sensor column 830 indicates “light”, and the microphone column 840 indicates “quiet”, the location is estimated to be “user's desk” in the location estimation column 850.

As shown in the example in the third row, in a case where the GPS column 810 indicates “office”, the six-axis sensor column 820 indicates “stationary”, the illuminance sensor column 830 indicates “light”, and the microphone column 840 indicates “noisy”, the location is estimated to be “meeting room” in the location estimation column 850.

As shown in the example in the fourth row, in a case where the GPS column 810 indicates “office”, the six-axis sensor column 820 indicates “walking”, the illuminance sensor column 830 indicates “dark”, and the microphone column 840 indicates “quiet”, the location is estimated to be “moving” in the location estimation column 850.

As shown in the example in the fifth row, in a case where the GPS column 810 indicates “being out (others)”, the six-axis sensor column 820 indicates “stationary”, the illuminance sensor column 830 indicates “light”, and the microphone column 840 indicates “noisy”, the location is estimated to be “cafe” in the location estimation column 850.

Alternatively, the location estimation table 800 may be configured by using a detection value obtained by a proximity sensor in the smartphone 200.

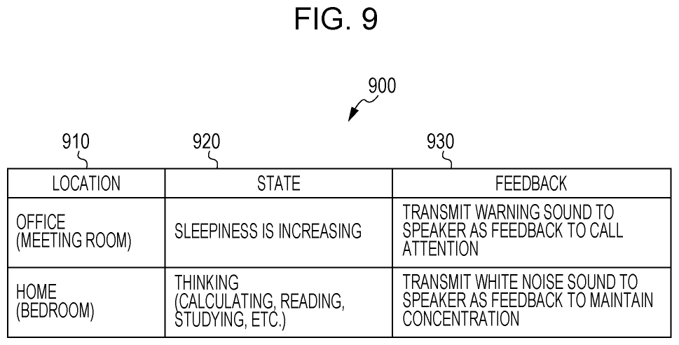

FIG. 9 is an explanatory diagram illustrating an example data structure of the feedback table 900.

The feedback table 900 includes a location column 910, a state column 920, and a feedback column 930. The location column 910 stores location data. The state column 920 stores state data. The feedback column 930 stores feedback data.

For example, as shown in the example in the first row of the feedback table 900, in a case where the location is “office (meeting room)” and the state is “sleepiness is increasing”, “transmit warning sound to speaker as feedback to call attention” is performed as feedback.

As shown in the example in the second row of the feedback table 900, in a case where the location is “home (bedroom)” and the state is “thinking (calculating, reading, studying, etc.)”, “transmit white noise sound to speaker as feedback to maintain concentration” is performed as feedback.

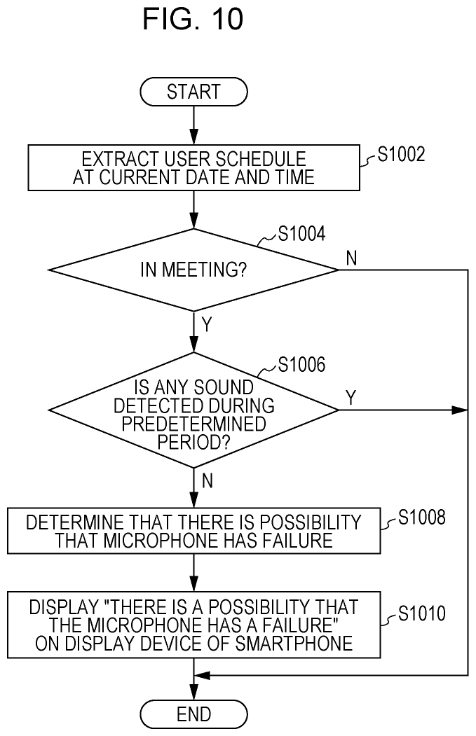

FIG. 10 is a flowchart illustrating an example of processing according to the exemplary embodiment.

The flowchart illustrated in FIG. 10 may be inserted between step S410 and step S412 in the flowchart illustrated in FIG. 4 or between step S710 and step S712 in the flowchart illustrated in FIG. 7.

In step S1002, a schedule at a current date and time of the user is extracted.

In step S1004, it is determined whether or not the user is in a meeting. In a case where the user is in a meeting, the processing proceeds to step S1006. Otherwise, the processing ends.

In step S1006, it is determined whether or not any sound is detected by the microphone 270 during a predetermined period. In a case where no sound is detected, the processing proceeds to step S1008. Otherwise, the processing ends.

In step S1008, it is determined that there is a possibility that the microphone 270 has a failure.

In step S1010, a message “there is a possibility that the microphone has a failure” is displayed on the display device of the smartphone 200.

After that, in step S412 in the flowchart illustrated in FIG. 4 or in step S712 in the flowchart illustrated in FIG. 7, the state of the user 300 is estimated by using brain wave information and action information. Specifically, the state of the user 300 may be estimated by using the state estimation table 500 except for the microphone column 530.

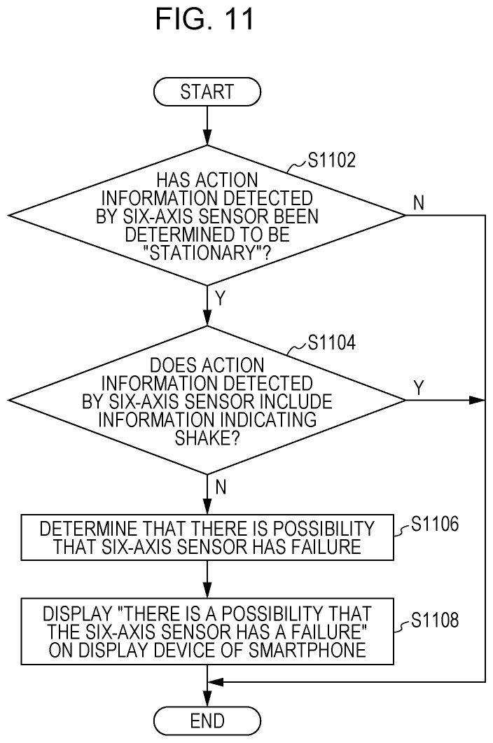

FIG. 11 is a flowchart illustrating an example of processing according to the exemplary embodiment.

The flowchart illustrated in FIG. 11 may be inserted between step S410 and step S412 in the flowchart illustrated in FIG. 4 or between step S710 and step S712 in the flowchart illustrated in FIG. 7.

In step S1102, it is determined whether or not the action information detected by the six-axis sensor 260 has been determined to be “stationary”. In a case where the action information has been determined to be “stationary”, the processing process to step S1104. Otherwise, the processing ends.

In step S1104, it is determined whether or not the action information detected by the six-axis sensor 260 includes information indicating a shake. In a case where the action information does not include information indicating a shake, the processing proceeds to step S1106. Otherwise, the processing ends.

In step S1106, it is determined that there is a possibility that the six-axis sensor 260 has a failure.

In step S1108, a message “there is a possibility that the six-axis sensor has a failure” is displayed on the display device of the smartphone 200.

After that, in step S412 in the flowchart illustrated in FIG. 4 or in step S712 in the flowchart illustrated in FIG. 7, the state of the user 300 is estimated by using brain wave information and sound information. Specifically, the state of the user 300 may be estimated by using the state estimation table 500 except for the action (six-axis) column 520.

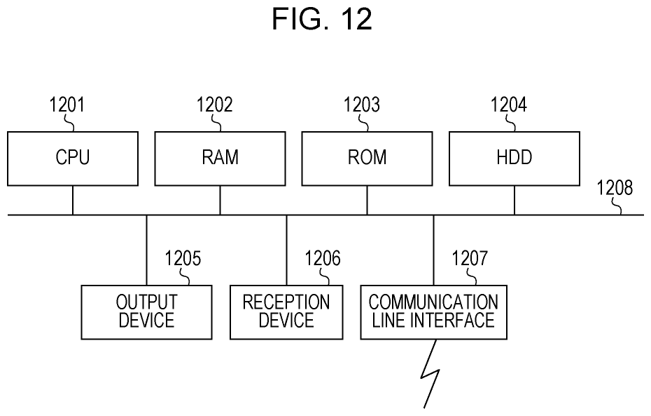

A hardware configuration of a computer that executes a program as the exemplary embodiment (the information processing device 100, the terminal 150, the smartphone 200, and the wearable device 250) is a typical computer as illustrated in FIG. 12 and is specifically a personal computer, a computer that can be a server, or the like. Specifically, a central processing unit (CPU) 1201 is used as a processing unit (computing unit), and a random access memory (RAM) 1202, a read only memory (ROM) 1203, and a hard disk drive (HDD) 1204 are used as a storage device. As the HDD 1204, an HDD, a solid state drive (SSD), which is a flash memory, or the like may be used, for example. The hardware configuration of the computer includes the CPU 1201 that executes a program of the communication module 105, the action information obtaining module 110, the brain wave information obtaining module 115, the sound information obtaining module 120, the analyzing module 125, the output control module 130, the communication module 155, the action detecting module 160, the brain wave detecting module 165, the sound detecting module 170, and the like; the RAM 1202 storing the program and data; the ROM 1203 storing a program or the like for activating the computer; the HDD 1204 serving as an auxiliary storage device having a function of storing data, a program, and the like; a reception device 1206 (the action detecting module 160, the brain wave detecting module 165, and the sound detecting module 170) that receives data in accordance with a user operation (including a motion, brain wave, sound, line of sight, and the like) performed on a keyboard, mouse, touch screen, microphone, camera (including a line-of-sight detecting camera or the like), or the like; an output device 1205 (the output device 175 and the output device 135), such as a cathode ray tube (CRT), a liquid crystal display, or a speaker; a communication line interface 1207 (the communication module 105 and the communication module 155) for connecting to a communication network, such as a network interface card; and a bus 1208 that connects these devices to transmit and receive data. Plural computers each having the above-described hardware configuration may be connected to each other through a network.

In the above-described exemplary embodiment, the processing based on a computer program is performed by cooperation between software and hardware resources by causing a system having the above-described hardware configuration to read the computer program as software. Accordingly, the above-described embodiment is carried out.

The hardware configuration illustrated in FIG. 12 is one example configuration. The exemplary embodiment is not limited to the configuration illustrated in FIG. 12 and may adopt any configuration capable of executing the modules described in the exemplary embodiment. For example, one or some of the modules may be constituted by dedicated hardware (for example, an application specific integrated circuit (ASIC), a reconfigurable integrated circuit (a field-programmable gate array (FPGA)), or the like), or one or some of the modules may be included in an external system and connected through a communication line. Furthermore, plural systems each having the hardware configuration illustrated in FIG. 12 may be connected to each other through a communication line and may operate in cooperation with each other. In particular, one or some of the modules may be incorporated in a mobile information communication device (including a mobile phone, a smartphone, a mobile device, a wearable computer, and the like), a home information appliance, a robot, a copier, a facsimile, a scanner, a printer, or a multifunction peripheral (an image processing device having functions of two or more of a scanner, a printer, a copier, a facsimile, and the like), as well as a personal computer.

The above-described program may be provided by storing it in a recording medium or may be provided through communication. In this case, for example, the above-described program may be regarded as a “computer-readable recording medium storing the program”.

The “computer-readable recording medium storing the program” is a computer-readable recording medium storing the program and used to install, execute, or distribute the program.

Examples of the recording medium include a digital versatile disc (DVD), such as “DVD-R, DVD-RW, DVD-RAM, and the like” defined by DVD Forum and “DVD+R, DVD+RW, and the like” defined by DVD+RW Alliance; a compact disc (CD), such as a read only memory (CD-ROM), a CD recordable (CD-R), and a CD rewritable (CD-RW); a Blu-ray Disc (registered trademark); a magneto-optical (MO) disc; a flexible disk (FD); magnetic tape; a hard disk; a read only memory (ROM); an electrically erasable and programmable ROM (EEPROM, registered trademark); a flash memory; a random access memory (RAM); and a secure digital (SD) memory card.

All or part of the above-described program may be stored or distributed by recording it on the recording medium. Alternatively, all or part of the program may be transmitted through communication, for example, using a transmission medium such as a wired or wireless communication network used in a local area network (LAN), a metropolitan area network (MAN), a wide area network (WAN), the Internet, an intranet, or an extranet, or a combination of the wired and wireless communication networks. Alternatively, all or part of the program may be carried using carrier waves.

Furthermore, the above-described program may be all or part of another program, or may be recorded on a recording medium together with another program. Alternatively, the program may be recorded on plural recording media in a split manner. The program may be recorded in any manner, for example, the program may be compressed or encrypted, as long as the program can be recovered.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing device comprising:
a first obtaining unit that obtains action information from a device worn on a head of a user, the action information being information indicating a motion of the head of the user;
a second obtaining unit that obtains biological information on the user from the device; and
an analyzing unit that analyzes, based on the action information and the biological information, a state of the user.

2. The information processing device according to claim 1, further comprising:
an output controller that performs control to output information to an output device included in the device in accordance with the state of the user analyzed by the analyzing unit.

3. The information processing device according to claim 2, wherein the output controller performs control to cause sound information corresponding to the state of the user to be output from the output device included in the device.

4. The information processing device according to claim 1, further comprising:
a third obtaining unit that obtains sound information from the device, the sound information being information indicating a sound produced by the user or a sound from surroundings of the user, wherein
the analyzing unit analyzes, based on the action information, the biological information, and the sound information, the state of the user.

5. The information processing device according to claim 4, wherein the analyzing unit obtains a schedule of the user, and in a case where the third obtaining unit does not obtain sound information at a time when a sound is supposed to be produced according to the schedule, analyzes that there is a possibility that a microphone included in the device has a failure.

6. The information processing device according to claim 4, wherein in a case where the action information obtained by the first obtaining unit does not include information indicating a shake of the head, the analyzing unit analyzes that there is a possibility that a motion detecting sensor included in the device has a failure.

7. The information processing device according to claim 6, wherein in a case where the analyzing unit analyzes that there is a possibility that the motion detecting sensor included in the device has a failure, the analyzing unit analyzes, based on the sound information and the biological information, the state of the user.

8. A non-transitory computer readable medium storing a program causing a computer to execute a process for information processing, the process comprising:
obtaining action information from a device worn on a head of a user, the action information being information indicating a motion of the head of the user;
obtaining biological information on the user from the device; and
analyzing, based on the action information and the biological information, a state of the user.

9. An information processing device comprising:
first obtaining means for obtaining action information from a device worn on a head of a user, the action information being information indicating a motion of the head of the user;
second obtaining means for obtaining biological information on the user from the device; and
analyzing means for analyzing, based on the action information and the biological information, a state of the user.
